(19) United States
(12) Patent Application Publication
(10) Pub. No.: US 2026/0015603 A1
(43) Pub. Date: Jan. 15, 2026

(54) A PROCESS AND CHROMATOGRAPHY MATERIAL FOR CHROMATOGRAPHY RECOVERY OF NUCLEIC ACID MOLECULES

(71) Applicant: Cytiva BioProcess R&D AB, Uppsala (SE)

(72) Inventors: Ronnie Palmgren, Uppsala (SE); Peter Lundback, Uppsala (SE); Birgit Blank, Uppsala (SE); Wangshu Jiang, Uppsala (SE); Susanna Lindman, Uppsala (SE); Ola Lind, Uppsala (SE); Nils Norrman, Uppsala (SE); Helen Cheek, Stevenage Hertfordshire (GB); Susana Brito Dos Santos, Stevenage Hertfordshire (GB); Per Denker, Uppsala (SE)

(21) Appl. No.: 18/992,539

(22) PCT Filed: Jul. 18, 2023
(86) PCT No.: PCT/EP2023/069907

§ 371 Date: Jan. 8, 2025

(30) Foreign Application Priority Data
Jul. 25, 2022 (SE) 2250928-5

(51) Int. Cl. C12N 15/10

(52) U.S. Cl. CPC C12N 15/101

(57) ABSTRACT

The present disclosure relates to a process for recovery of a nucleic acid product from a composition. The process (100) comprising: (i) contacting (110) the composition with a chromatography material functionalised with a ligand. The chromatography material comprises nanofibers; (ii) optionally washing (120) the functionalised chromatography material with a washing liquid phase; (iii) selectively eluting (130) said product by contacting the functionalised chromatography material with an elution liquid phase; (iv) cleaning-in-place (140) comprising regenerating the chromatography material by contacting with a cleaning liquid phase; (v) repeating steps (i)-(iii) for at least 15 cycles, wherein step (iv) is performed in at least one of said cycles; and (vi) collecting (150) recovered nucleic acid product. The chromatography material being capable of retaining a dynamic binding capacity at 10% breakthrough for said product after 50 cycles that is at least 80% of the corresponding dynamic binding capacity of the first cycle.

### TECHNICAL FIELD

The invention relates to processes for purification nucleic acid molecules, such as polyadenylated products like mRNA from biological compositions. The process involves contacting the composition functionalized chromatography material comprising a convection-based chromatography material.

### BACKGROUND

Messenger RNA (mRNA) is the key mediator in the central dogma of molecular biology. Single-stranded mRNA is transcribed from and is complementary to one of the DNA strands of a gene and its protein coding region specify the amino acid sequence of the protein. Prior to its role as a protein encoding template, pre-mRNA is processed to mRNA through a series of events that mainly occur in the nucleus either post-transcriptionally or concomitantly with the transcription from the DNA gene template. These critical events include 5′-capping, intron splicing, polyadenylation of the 3′ end, and shuttling from nucleus to the cytoplasm. All these features and events serve its own purpose and is critical for overall mRNA stability and modulation of translational efficiency. The 5′ cap and the polyA tail are both unique features of mRNA.

Technology advances in the synthetic mRNA field have put synthetic mRNA in the spotlight and is currently evaluated in several preclinical and clinical studies for a variety of diseases.

The production of synthetic mRNA by in vitro transcription (IVT) involves the key components 1) DNA template, 2) ribonucleotides and 3) RNA polymerase. The UTR sequences and protein coding sequence are defined by the DNA template. The 3′ polyA tail may or may not be designed within the DNA template. If included in the DNA template, the length of the polyA tail is more controllable while post-IVT polyA-tailing with a polyA polymerase may be used when not designed within the DNA template. Several different 5′-capping analogues and strategies exists, both co- and post-transcriptional approaches.

Oligo(dT) products are commonly used in ‘open purifications systems’ such as magnetic particles or spin columns. During hybridization (binding phase), the mRNA polyA-tail hybridizes to the oligo(dT) ligand in high salt buffers. The high conductivity limits electrostatic repulsion of the negatively charged backbones of the polyA and oligo(dT) ligand. This is typically, followed by washing and a mild elution using low conductivity buffers or water that destabilizes the TA pair and allows elution. Undesired contaminants such as proteins, unreacted ribonucleotides, DNA, CAP analogues and partial transcripts that lack the polyA moiety are not retained on the solid support during the hybridization or wash phase.

The length of the oligo(dT) ligand for the magnetic products usually ranges between 14-30 nucleotides. A small particle size provides a large particle surface area per mL and the size of the particles commonly ranges between 1-5 μm. The protocol length for small-scale mRNA purification is generally shorter than 1 h. Noteworthy, these products were designed and are generally used for purification of an mRNA pool from cell lysates and not for purification of mRNA from an IVT reaction. In addition, the known products are designed to operate in microcentrifuge tubes and given their small size, and consequently low magnetism, it is very unlikely that any of these products are scalable for processing larger sample volumes. In addition, such small particles packed as a chromatography material would have significantly impaired flow properties as compared to more conventional chromatography resins.

Furthermore, there are several drawbacks using known adsorbent materials for chromatographic mRNA separations.

Separations involving membranes and monoliths can be run at far higher flowrates than porous bead-based systems, typical residence times being in the order of 0.2-0.5 minutes. However, typical binding capacities at 10% breakthrough of target for monoliths and membranes under dynamic flow are lower than porous beads. The difference in binding capacity of monolith and membrane materials (compared to porous bead-based materials) may be offset by utilising higher flowrates. In membrane adsorption chromatography, in contrast to gel-permeation chromatography, there is binding of components of a fluid, for example individual molecules, associates or particles, to the surface of a solid in contact with the fluid without the need for transport in pores by diffusion and the active surface of the solid phase is accessible for molecules by convective transport. The advantage of membrane adsorbers over packed chromatography columns is their suitability for being run with much higher flow rates.

This is also called convection-based chromatography. A convection-based chromatography matrix includes any matrix in which application of a hydraulic pressure difference between the inflow and outflow of the matrix forces perfusion of the matrix, achieving substantially convective transport of the substance(s) into the matrix or out of the matrix, which is effected very rapidly at a high flow rate.

Convection-based chromatography and membrane adsorbers are described in for example US20140296464A1, US20160288089A1, US2019308169A1 and US2019234914A1, hereby incorporated by reference in their entireties.

There exists a need for improved solutions to separate nucleic acids, such as separating mRNA from an in vitro transcribed (IVT) reaction to enable a therapeutic product to be recovered at industrial scale.

### SUMMARY

One object of the invention is to provide improved solutions to recover nucleic acid products from a composition.

This has in accordance with the present disclosure been achieved by means of a process for recovery of a nucleic acid product from a composition comprising said product. The process comprising the steps of:
(i) contacting the composition with a chromatography material under conditions allowing nucleic acid base-pairing between at least part of the nucleic acid product and a ligand, wherein the chromatography material comprises one or more nanofibers, and wherein the chromatography material is functionalised with said ligand;
(ii) optionally washing the functionalised chromatography material with a washing liquid phase;
(iii) selectively eluting said product by contacting the functionalised chromatography material with an elution liquid phase;
(iv) cleaning-in-place comprising regenerating the chromatography material by contacting with a cleaning liquid phase;
(v) repeating steps (i)-(iii) for at least 15 cycles, wherein step (iv) is performed in at least one of said cycles; and
(vi) collecting recovered nucleic acid product.

The chromatography material being capable of retaining a dynamic binding capacity at 10% breakthrough for said nucleic acid product after 50 cycles that is at least 80% of the corresponding dynamic binding capacity of the first cycle.

This has the advantage of allowing use of a chromatography material with high binding capacity and suitable for higher flowrates. This further has the advantage of allowing large nucleic acid molecules, such as mRNA, to have relatively high probability of accessing binding areas of chromatography materials even during short residence times. The short residence times compared to traditional chromatography solid supports reduce the time of sample loading allows for more rapid purification of the desired nucleic acid that is especially important for sensitive biomolecules such as mRNA. This and even further in combination with a high capacity of the chromatography material will improve overall process productivity and economy and accelerate process development. In addition, such a process is especially well suited in a continuous chromatography system setup.

In some embodiments, steps (i)-(iii) are repeated at least 25 cycles, or at least 50 cycles.

In some embodiments, said nucleic acid product is single-stranded RNA, double-stranded RNA, mRNA, pre-mRNA, single-stranded DNA, and/or double-stranded DNA.

The ligand may be a nucleic acid capable of complementary base-pairing via a portion of the nucleic acid product of interest. In embodiments, said ligand is arranged to base pair via at least 10 bases of said nucleic acid product. The ligand may be attached to the nanofibers of the chromatography material.

In some embodiments, the chromatography material is functionalised with oligo(dT) ligands, and wherein the oligo(dT) ligand is a (dT)₁₀₋₅₀ ligand, preferably a (dT)₁₂₋₃₀ ligand, and wherein the nucleic acid product comprises a polyA-tagged product.

In some embodiments, said chromatography material comprises non-woven material comprising polymer nanofibers.

This has the advantage of allowing convective flow and direct mass transfer of the nucleic acid product to the ligand, thus resulting in faster kinetics of target-ligand interaction. Consequently, shorter contact times are needed for processing of a composition in comparison to typical porous resins.

The present disclosure further relates to a chromatography material. Said material is convection-based and is functionalized with ligands comprising an oligonucleotide. Said material comprises polymer nanofibers and may be in the form of one or more non-woven membrane(s) and/or sheet(s). Said ligands, which may be attached to the nanofibers, are arranged to base pair with at least part of a nucleic acid product.

In some embodiments, the chromatography material is capable of retaining a dynamic binding capacity at 10% breakthrough for said nucleic acid product after 50 repeated nucleic acid product-binding and elution events that is at least 80% of the corresponding dynamic binding capacity of the first cycle.

### BRIEF DESCRIPTION OF THE DRAWINGS

FIG. 1 depicts schematically a process for repeatedly recovering a nucleic acid product from a composition.

FIG. 2 is a graph showing the dynamic binding capacity (DBC) for mRNA of different length on a chromatography material functionalized with oligo (dT) ligands (Fibro).

FIG. 3 is a graph showing dynamic binding capacity and ligand density for two different ligand lengths, (dT)₃₀ and (dT)₂₀, respectively.

FIG. 4 is a graph showing the impact on dynamic binding capacity of two different linkers, C12 and C6, using the same ligand length as in FIG. 3.

FIG. 5 is a graph showing the pressure profile, as described in the Example section, for 10 consecutive cycles without cleaning-in-place (CIP).

FIG. 6 depicts a chromatogram depicting the conductivity/absorbance and dynamic binding results for a series of cycles at different residence times.

FIG. 7 depicts a plot of dynamic binding capacity vs retention time for the series of cycles in FIG. 6.

FIG. 8A-B show chromatograms depicting the conductivity/absorbance and dynamic binding results for subsequent cycles.

FIG. 9A-C depicts experimental and estimated dynamic binding capacity changes after multiple cycles.

FIG. 10 is a graph showing the dynamic binding capacity DBC for differently sized mRNAs at various KCl concentrations during binding.

FIG. 11 is a graph showing the dynamic binding capacity over 200 cycles of cleaning-in-place.

### DETAILED DESCRIPTION

The invention will now be described more closely in relation to some non-limiting Examples and the accompanying figures.

Throughout the figures, same reference numerals refer to same parts, concepts, and/or elements. Consequently, what will be said regarding a reference numeral in one figure applies equally well to the same reference numeral in other figures unless not explicitly stated otherwise.

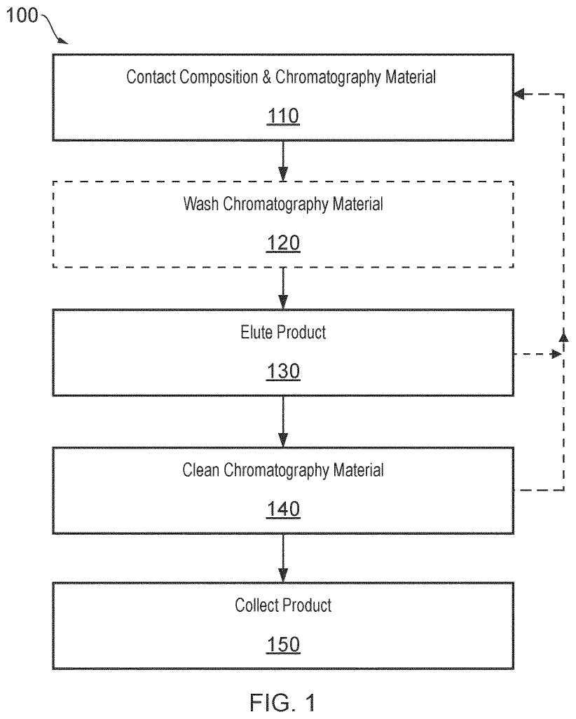

FIG. 1 depicts schematically an example process for recovery of a nucleic acid product from a composition comprising said product, the process 100 comprising the steps of:
(i) contacting 110 the composition with a chromatography material under conditions allowing nucleic acid base-pairing between at least part of the nucleic acid product and a ligand, wherein the chromatography material comprises one or more nanofibers, preferably cellulose nanofibers, wherein the chromatography material is functionalised with said ligand;
(ii) optionally washing 120 the functionalised chromatography material with a washing liquid phase;
(iii) selectively eluting 130 said product by contacting the functionalised chromatography material with an elution liquid phase; and
(iv) cleaning-in-place 140 comprising regenerating the chromatography material with a cleaning liquid phase;
(v) repeating steps (i)-(iii) for at least 15 cycles, wherein step (iv) is performed in at least one of said cycles;
(vi) collecting 150 the recovered nucleic acid product,
   wherein the chromatography material is capable of retaining a dynamic binding capacity at 10% breakthrough for said nucleic acid product after 50 cycles which is at least 80% of the corresponding dynamic binding capacity of the first cycle.

In FIG. 1 the dashed lines connecting the eluting 130 step and the cleaning-in-place step 140 step with the contacting 110 step represents starting a new cycle with the option of omitting the cleaning-in-place 140 step in some cycles. Collecting 150 the recovered nucleic acid product is not dependent on a cycle performing the cleaning-in-place 140 step. The step of collecting 150 the recovered nucleic acid product relates to collecting recovered nucleic acid product for the whole process. For example, collecting 150 the recovered nucleic acid product may comprise collecting the eluate of the step of selectively eluting 130 from all cycles.

It is to be understood that the expression “repeating steps (i)-(iii)” entails performing the step of contacting 110 and the step of selectively eluting 130, with the step of washing 120 being optional.

In some examples, the at least one criteria comprises that the dynamic binding capacity for said nucleic acid product of the last cycle of said at least 50 cycles is at least 80% of the dynamic binding capacity of the first cycle.

The expression “washing the functionalised chromatography material” relates to washing 120 the chromatography material between contacting 110 and elution 130.

In some examples, instead of step (iv) being performed in at least one of said cycles, the step of cleaning-in-place 140 is an optional step. In some examples, the step of cleaning-in-place 140 is performed between each cycle. Typically, cleaning-in-place 140 is performed after selectively eluting 130.

The term “regeneration” of a chromatography material means herein a process that substantially restores or attempts to restore the chromatography material to its original strength or properties.

It is to be understood that cleaning-in-place, CIP, may be performed in a multitude of ways with different combinations of buffer, chemicals, solvents, temperatures and durations. The expression “cleaning-in-place” of the chromatography material relates to cleaning and/or regenerating the chromatography material with the cleaning liquid phase.

In some examples, contacting 110 the composition with the chromatography material comprises the composition being having a residence time in contact with the chromatography material for a period of 10-15 seconds. In some of these examples, for a period of 5-20 seconds, for a period of 3-25 seconds, for a period of 1-35 seconds, or for a period of 8-45 seconds.

In some examples, contacting 110 the composition with the chromatography material has a residence time between 5 s to 300 s. In some of these examples, the residence time is between 15 s to 120 sec. In some examples, contacting 110 the composition with the chromatography material is performed at ambient temperature. In some examples, contacting 110 the composition with the chromatography material is performed during salt concentrations between 100 mM to 1000 mM. In some of these examples, the electrolyte comprises KCl, NaCl or other salts with equivalent/comparable conductivity, such as NaCl. In some examples, said salt concentration is below 500 mM. In some of these examples, said salt concentration is below 100 mM, below 150 mM, below 200 mM, below 250 mM, below 300 mM, below 350 mM, below 400 mM, below 450 mM, or below 600 mM.

In some examples, contacting 110 the composition with a chromatography material comprises utilizing a composition with said product in concentration ranges between 0.1-10 mg/mL. In some of these examples, between 1-5 mg/ml, or between 2-20 mg/mL.

In some examples, contacting 110 the composition with a chromatography material comprises DNase I treating and/or Proteinase K treating the composition before contacting with the chromatography material. In some examples, the composition comprises a chelator, such as ethylenediaminetetraacetic acid, EDTA.

In some examples, the composition comprises KCl 200 mM, Tris 10 mM, and EDTA 1 mM and has a pH of about 7.5.

In some examples, said composition comprises a nucleic acid product from an in vitro transcribed (IVT) reaction. In some of these examples, said composition comprises solutions originating from or obtainable from cell culture, such as cell lysates, cell culture supernatant. In some examples, contacting 110 the composition comprises a prior separation and/or purification of said solutions originating from or obtainable from cell culture to obtain said nucleic acid product comprised in said composition or parts thereof.

In some examples, said composition is of enzymatic, synthetic and/or biological origin. It is to be understood that the origin will impact process in terms of impurities, contaminants and yields. Typically, the process of the invention allows for relatively short residence times to be utilized to recover a corresponding product independent of the composition origin, however, the process may benefit from adjustments based on the origin of the composition.

In some examples, said chromatography material comprises one or more polymer nanofibers.

In some examples, said chromatography material comprises non-woven material comprising polymer nanofibers. In some of these examples, said polymer nanofibers comprise cellulose nanofibers.

The term nanofiber relates to fibers with diameters in the nanometre range. Typically, nanofibers have diameters between 1 nm and 1 μm.

In some examples, said chromatography material comprises an open structure and is configured to allow convective flow of a composition through said open structure. Convective flow allows for direct mass transfer of the product to the ligand, thereby providing faster kinetics of target-ligand interaction. Consequently, the open structure results in shorter contact times being required for processing of the composition in contrast to typical porous resins.

The chromatography medium may have a mean flow pore size of 0.1-2.0 μm. Mean flow pore (MFP) size is an indicator of material flow characteristics, and is measured by capillary flow porometry, based on the displacement of a wetting liquid with a known surface tension from the sample pores by applying a gas at increasing pressure, for example as described in Example 2 below. The higher the MFP size, the larger the flow of liquid through the material at a given pressure. The mean flow pore size is calculated from the point at which 50% of the flow goes through a sample. Mean flow pore size thus corresponds to the pore size calculated at the pressure where the wet curve and the half-dry curve meet.

In an alternative definition, the mean flow pore size of the present matrix material may be seen as an effective pore size defined as the size of the largest sphere that is able to pass through the pore.

The mean flow pore size of the matrix material may be 0.1-2.0 μm, 0.1-1.8 μm, 0.1-1.6 μm, 0.1-1.4 μm, 0.1-1.2 μm, 0.1-1.0 μm, 0.1-0.8 μm, 0.1-0.6 μm, 0.1-0.4 μm, 0.1-0.2 μm, 0.2-2.0 μm, 0.4-2.0 μm, 0.6-2.0 μm, 0.8-2.0 μm, 1.0-2.0 μm, 1.2-2.0 μm, 1.4-2.0 μm, 1.6-2.0 μm, 1.8-2.0 μm, or 0.5-1.5 μm.

In some examples, said chromatography material is functionalized with ligands of a ligand density of 3 to 20 μmole/g. In some examples, the ligand density is 1 to 10 μmole/g, 0.5 to 5 μmole/g, or 2 to 30 μmole/g.

In some examples, said ligand is arranged to base pair via at least 5 bases with said nucleic acid product. In some of these examples, said ligand is arranged to base pair via at least 10 bases, at least 15 bases, at least 25 bases, at least 50 bases, at least 100 bases, or at least 200 bases. In some of these examples, said ligand is arranged to base pair via at most 10 bases, at most 15 bases, at most 25 bases, at most 50 bases, at most 100 bases, or at most 200 bases.

In some examples, said ligand comprises an oligonucleotide arranged to base-pair with at least a portion of said nucleic acid product.

In some examples, the ligand is coupled to the chromatography material via a linker, such as such as a C6 or C12 alkyl linker comprising a carbon backbone. In some of these examples, said C6 linker comprises a (CH₂)₆ spacer, and correspondingly said C12 linker comprises a (CH₂)₁₂ spacer. In some examples, the ligand is coupled to the chromatography material via a linker comprising a carbon backbone between 3 to 18 carbon long. In some embodiments, the linker has a length corresponding to the length of a carbon backbone having 6-12 carbon atoms.

In some examples, said elution liquid phase has a salt concentration of between 0 to 50 mM. In some of these examples, said salt concentration is between 0 to 10 mM, 0 to 20 mM, 0 to 30 mM, or 0 to 40 mM. Typically, said salt concentration is preferably close to 0 mM.

In some examples, said elution liquid phase has a pH value in the range between pH 5.5 to pH 9. In some examples, said elution liquid phase is a buffer and/or aqueous solution, such as pure water or any buffer in the range of pH 5.5-9.

In some examples, said elution liquid phase comprises Tris, Hepes, sodium phosphate, and/or sodium citrate. In some examples, said elution liquid phase comprises a chelator, such as EDTA. In some examples, said elution liquid phase comprises Tris 10 mM, and EDTA 1 mM@pH 7.5.

Typically, the salt concentration and/or total ionic concentration in the liquid phase at the chromatography material decreases significantly during elution 130.

In some examples, washing 120 the functionalised chromatography material is performed at ambient temperature.

In some examples, washing 120 is performed in at least a third of the cycles, or at least half of the cycles. In some of these examples, washing 120 is performed every cycle.

In some examples, the composition has a salt concentration below 500 mM. In some examples, the composition has a salt concentration below 400 mM, below 300 mM or below 200 mM. In some of these examples, the composition comprises KCl, NaCl, or other salts with equivalent/comparable conductivity.

In some of these examples, the washing liquid phase comprises KCl, NaCl or other salts with equivalent/comparable conductivity.

It is to be understood that the washing liquid phase utilized during washing 120 may be the same buffer system as used during contacting 110 the composition with said chromatography material.

In some examples, the washing liquid phase has a lower salt concentration than the composition during contacting 110, thereby facilitating the following elution step.

In some examples, the washing liquid phase has lower conductivity than said buffer system used during contacting 110.

In some examples, steps (i)-(iii) are repeated for at least 20 cycles. In some of these examples the number of repeated cycles are at least 30, at least 40, at least 50, at least 60, at least 80, at least 100, at least 125, at least 150, at least 175, or at least 200.

In some examples, the chromatography material is capable of retaining a dynamic binding capacity at 10% breakthrough for said nucleic acid product after 50 cycles that is at least 80% of the corresponding dynamic binding capacity of the first cycle according to the method for determining dynamic binding capacity as described herein. In some of these examples, the dynamic binding capacity ratio against the first cycle is at least 70%, at least 75%, at least 85%, at least 90%, or at least 95%.

In some examples, the chromatography material is capable of retaining a dynamic binding capacity at 10% breakthrough for said nucleic acid product after 60 cycles that is at least 80% of the corresponding dynamic binding capacity of the first cycle. In some of these examples, dynamic binding capacity at 10% breakthrough for said nucleic acid product at the first cycle is compared to the corresponding value after 15 cycles, 20 cycles, 30 cycles, 40 cycles, 70 cycles, 80 cycles, 100 cycles, 125 cycles, 150 cycles, or 200 cycles.

It is to be understood that the above examples for the dynamic binding capacity criteria and the examples the cycle for which a comparison is made may be combined freely. For example disclosing an example chromatography material capable of retaining a dynamic binding capacity at 10% breakthrough for said nucleic acid product after 80 cycles that is at least 75% of the corresponding dynamic binding capacity of the first cycle.

In some examples, cleaning-in-place 140 is performed at least once every other cycle, once every third cycle, once every fourth cycle, once every five cycles, once every six cycles, once every seven cycles, once every eight cycles, once every nine cycles, once every ten cycles, once every twenty cycles, once every thirty cycles and/or once every fifty cycles. Typically, the optimal frequency for cleaning-in-place will depend on the material and cost as the cleaning-in-place 140 step increases the duration of the cycle.

In some examples, cleaning-in-place 140 is performed at most once every other cycle, once every other cycle, once every third cycle, once every third cycle, once every fourth cycle, once every five cycles, once every six cycles, once every seven cycles, once every eight cycles, once every nine cycles, once every ten cycles, once every twenty cycles, once every thirty cycles and/or once every fifty cycles. In some examples, cleaning-in-place 140 is performed once every 3 to 9 cycles.

Clean-in-place relates to a cleaning step performed without the need to disassemble a unit, such as a chromatography system or holders of the chromatography material. Clean-in-place relates to soaking, filling and/or flowing a solvent, buffer, and/or chemical into contact with a solid phase to be cleaned and/or regenerated, such as the chromatography material, for a duration that ensures that the functionalized solid phase is sufficiently cleaned to limit cycle-to-cycle contamination and restores its functionality.

The expression “regenerating the chromatography material” is to be understood as performing an action attempting to restore an initial functionality of said chromatography material, however, typically such actions do not result in full restoration of functionality.

For example, cleaning-in-place may comprise NaOH, and/or chaotropic agents such as Guanidine-HCl or urea. In some examples, cleaning-in-place utilizes a cleaning liquid phase comprising a NaOH and/or an isopropanol solution. In some examples, cleaning-in-place utilizes peracetic acid.

In some examples, said cleaning liquid phase comprises NaOH 100 mM. In some examples, said cleaning liquid phase comprises between 10 mM and 1 M NaOH.

In some examples, said cleaning liquid phase comprises sodium hydroxide, nitric acid, phosphoric acid and/or sulphuric acid.

In some examples, said cleaning liquid phase has at least pH 8. In some examples, said cleaning liquid phase has at least pH 9, at least pH 10, at least pH 11, at least pH 12, or at least pH 13.

In some examples, collecting 150 the recovered nucleic acid product comprises collecting at least part of the contacted elution liquid phase. Typically, the step of collecting 150 the recovered nucleic acid product follows a standard procedure for a used chromatography system.

An example process 100 for recovering a polyA-tagged product from a composition comprising said product. The process 100 comprising the steps of:
(i) contacting 110 the composition with a chromatography material comprising one or more non-woven material of polymer nanofibers, preferably cellulose nanofibers, the chromatography material being functionalised with oligo(dT) ligands, wherein the oligo(dT) ligand is a (dT)10-50 ligand, preferably a (dT)12-30 ligand;
(ii) optionally washing 120 the functionalised chromatography material with a washing liquid phase;
(ii) selectively eluting 130 the polyA-tagged product by contacting the functionalised chromatography material with an elution liquid phase of significantly lower salt concentration than the composition; and
(iv) repeating steps (i)-(iii) for at least 30 cycles, wherein step (ii) is performed in at least one cycle, and wherein the chromatography material performance for the last cycle of said at least 30 cycles and the dynamic binding capacity for said polyA tagged product of the last cycle of said at least 30 cycles is at least 80% of the dynamic binding capacity of the first cycle.

Another example process is for recovering a polyA-tagged product from a composition comprising said product, the process 100 comprising the steps of:
(i) contacting 110 the composition with a chromatography material under conditions allowing base-pairing between the polyA tag and the oligo(dT), wherein the chromatography material comprises one or more polymer nanofibers, preferably cellulose nanofibers, wherein the chromatography material is functionalised with oligo(dT) ligands, and wherein the oligo(dT) ligand is a (dT)₁₀₋₅₀ ligand, preferably a (dT)₁₂₋₃₀ ligand;
(ii) optionally washing 120 the functionalised chromatography material with a washing liquid phase;
(iii) selectively eluting 130 the polyA-tagged product by contacting the functionalised chromatography material with an elution liquid phase;
(iv) cleaning-in-place 140 comprising regenerating the chromatography material with a cleaning liquid phase; and
(iv) repeating steps (i)-(iii) for at least 50 cycles, wherein step (iv) is performed in at least one cycle,
(vi) collecting 150 the recovered polyA-tagged product, and
   wherein the chromatography material is capable of retaining a dynamic binding capacity at 10% breakthrough for said polyA-tagged product after 50 cycles which is at least 80% of the corresponding dynamic binding capacity of the first cycle.

The chromatography material utilized in the process of the present invention comprises convection-based chromatography material. A convection-based chromatography material can be for example an adsorptive membrane where a flow through such materials is convective rather than diffusional. In some examples, the adsorptive membrane is, or comprises, a polymer nanofiber membrane, such as for example cellulose, cellulose acetate and/or cellulose fibres that have been treated for use as an adsorbent. In some examples, the adsorptive membrane is, or comprises, a monolithic material and/or a conventional membrane made by emulsification. Another alternative is a 3D printed material.

In some examples, the adsorptive membrane comprises polymer nanofibers. Typically, the polymer nanofibers are in the form of one or more non-woven sheets, each sheet comprising one or more said polymer nanofibers. A non-woven sheet comprising one or more polymer nanofibers is a mat of said one or more polymer nanofibers with each fibre oriented essentially randomly, i.e. it has not been fabricated so that the fibre or fibres adopts a particular pattern. Non-woven sheets comprising polymer nanofibers are typically provided by known methods. Non-woven sheets may, in certain circumstances, consist of a single polymer nanofiber. Alternatively, non-woven sheets may comprise two or more polymer nanofibers, for example 2, 3, 4, 5, 6, 7, 8, 9 or 10 polymer nanofibers.

In some examples, the chromatography material comprises one or more polymer nanofibers. In some examples, the chromatography material comprises a non-woven material comprising polymer nanofibers.

Preferably, the utilized chromatography material is in the form of one or more membrane(s) or sheet(s) and the composition is passed through a holder comprising one or more said membranes or sheets and optionally one or more frits or other spacer materials.

Optionally a heatable metal structure is arranged between the membranes or sheets of said chromatography material that facilitates the elution of the polyA-tagged product when the device is heated.

In some examples, said polymer nanofibers comprises electrospun polymer nanofibers. Such electrospun polymer nanofibers are well known to the person skilled in the art. Alternative methods for producing polymer nanofibers may also be used, e.g. drawing.

Polymer nanofibers for use in the present invention typically have mean diameters from 10 nm to 1000 nm. For some applications, polymer nanofibers having mean diameters from 200 nm to 100 nm are appropriate. Polymer nanofibers having mean diameters from 200 nm to 400 nm may be appropriate for certain applications.

The length of polymer nanofibers for use in the present invention is not particularly limited. Thus, conventional processes e.g. electrospinning can produce polymer nanofibers many hundreds of metres or even kilometres in length. Typically, though, the one or more polymer nanofibers have a length up to 10 km, preferably from 10 m to 10 km.

Non-woven sheets typically have area densities from 1 to 40 g/m². In some examples, area densities are from 5 to 25 g/m². In some examples, area densities are from 1 to 20 g/m², or 5 to 15 g/m².

Non-woven sheets typically have a thickness from 5 to 120 μm. In some examples, thickness is from 10 to 100 μm. In some examples, thickness is from 50 to 90 μm, 5 to 40 μm, 10 to 30 μm, or 15 to 25 μm.

The polymer used to produce the nanofibers used in the processes of the present invention is not particularly limited, provided the polymer is suitable for use in chromatography applications. Thus, typically, the polymer is a polymer suitable for use as a chromatography material, i.e. an adsorbent, in chromatography. Suitable polymers include polyamides such as nylon, polyacrylic acid, polymethacrylic acid, polyacrylonitrile, polystyrene, polysulfones e.g. polyethersulfone (PES), polycaprolactone, collagen, chitosan, polyethylene oxide, agarose, agarose acetate, cellulose, cellulose acetate, and combinations thereof. Polyethersulfone (PES), cellulose and cellulose acetate are preferred. In some cases, cellulose and cellulose acetate are preferred.

In some examples, the polymer used to produce the nanofibers used in the processes comprises, or consists of, nylon, polyacrylic acid, polymethacrylic acid, polyacrylonitrile, polystyrene, polysulfones e.g. polyethersulfone (PES), polycaprolactone, collagen, chitosan, polyethylene oxide, agarose, agarose acetate, cellulose, cellulose acetate, and/or combinations thereof.

Typically, the functionalised chromatography material used in the processes is a functionalised cellulose chromatography material. Preferably, the functionalised chromatography material is formed of one or more non-woven sheets, each comprising one or more cellulose or cellulose acetate nanofibers. Cellulose acetate is readily formed into nanofibers, e.g. by electrospinning and can readily be transformed into cellulose after electrospinning.

In a preferred embodiment, the functionalised chromatography material used in the processes comprises one or more polymer nanofibers. In another embodiment, the functionalised chromatography material used in the processes comprises one or more of any type of polymer fibre. Said polymer fibres may have any or all of the same properties as the nanofibers described above. Typically, said polymer fibres may have mean diameters from 10 nm to 1000 μm, preferably from 10 nm to 750 μm, more preferably from 10 nm to 500 μm, even more preferably from 10 nm to 400 μm, even more preferably from 10 nm to 300 μm, even more preferably from 10 nm to 200 μm, even more preferably from 10 nm to 100 μm, even more preferably from 10 nm to 75 μm, even more preferably from 10 nm to 50 μm, even more preferably from 10 nm to 40 μm, even more preferably from 10 nm to 30 μm, even more preferably from 10 nm to 20 μm, even more preferably from 10 nm to 10 μm, even more preferably from 10 nm to 5 μm, even more preferably from 10 nm to 4 μm, even more preferably from 10 nm to 3 μm, even more preferably from 10 nm to 2 μm, even more preferably from 10 nm to 1 μm (1000 nm).

In some examples, the ligand arranged to bind nucleic acid products from a composition is an oligo(dT) ligand arranged to bind to polyadenylated (polyA-tagged) mRNA.

In some examples, the process utilizes nanofibers functionalised with oligo(dT) ligand, such as a (dT)₁₀₋₅₀ ligand, or preferably a (dT)₁₂₋₃₀ ligand.

Use of multiple non-woven sheets of polymer nanofibers enables a thicker material to be prepared which has a greater capacity for adsorption. The functionalised chromatography material is typically therefore formed by providing two or more non-woven sheets stacked one on top of the other, each said sheet comprising one or more polymer nanofibers, and simultaneously heating and pressing the stack of sheets to fuse points of contact between the nanofibers of adjacent sheets.

Preferred chromatography material production conditions for pressing and heating of polymer nanofibers/non-woven sheets can be found in WO-A-2015/052460 and WO-A-2015/052465, the entirety of which are incorporated herein by reference.

The functionalised chromatography material used in the process has a dynamic binding capacity (DBC) that is dependent of the size of the mRNA and specific examples are given in the examples below. The DBC for 10% breakthrough can be determined in accordance with standard means, e.g. using an AKTA Pure system or equivalent FPLC systems.

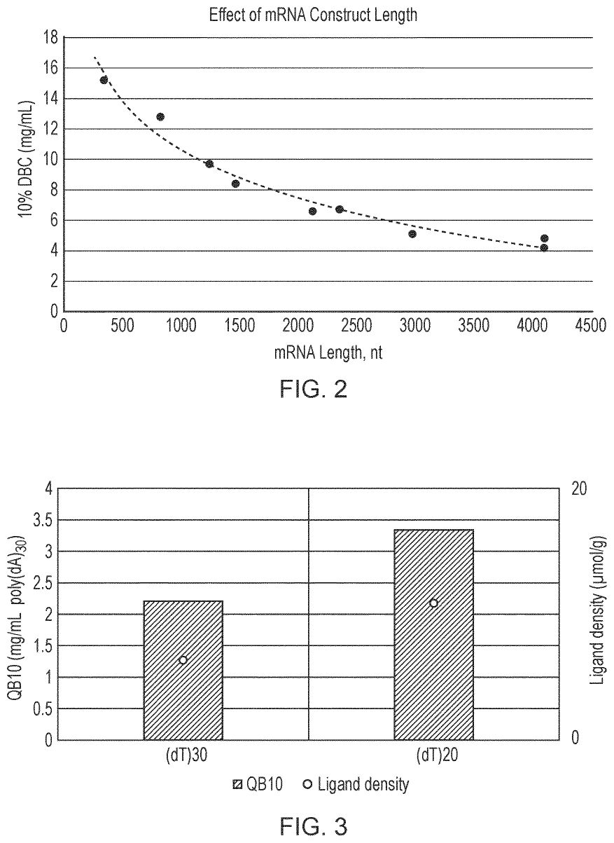

Typically, the nucleic acid product, e.g. polyA-tagged product, comprised in the composition is a nucleic acid with size in the range between 200-50000 bases. For example, DBC 4-16 mg/ml is expected for 4000-500 nucleotide polyA-tagged RNA, as shown in FIG. 2.

DBC for 10% breakthrough is typically determined according to the following assay method:
1) Loading material is passed through functionalised material contained within a holder on an AKTA Pure system (Cytiva);
2) Material is loaded under a determined matrix volume per minute flowrate (mV/min) until the concentration after the holder outlet exceeded 10% of that loaded as determined by the UV flow cell;
3) Accounting for dead volumes in the system and the holder device the total amount of protein loaded onto the disc at the 10% breakthrough was determined through analysis of the chromatogram in the Unicorn software (Cytiva).

Whether or not the chromatography material is capable of retaining a DBC at 10% breakthrough of at least 80% after 50 cycles can be determined by 1) performing a first binding cycle on fresh (not previously used) chromatography material and measuring the DBC at 10% breakthrough 2) performing 50 chromatography cycles as described herein, and 3) measuring the DBC at 10% breakthrough. If the value of the DBC determined in step 3) represents at least 80% of the value of the DBC obtained in step 1), the material is considered capable of retaining a DBC at 10% breakthrough of at least 80% after 50 cycles.

Insofar the process parameters are the same for the DBC determinations of steps 1) and 3), the actual values of the parameters are not crucial, and can be selected within the normal operating range by a person of skill in the art.

In some examples, the process utilizes functionalised chromatography material housed in a chromatography cartridge and/or holder. The cartridge typically comprises one or more functionalised chromatography media utilized in the present invention. The cartridge is typically cylindrical.

Typically, the used chromatography cartridge comprises one or more functionalised chromatography media stacked and/or wound inside a typically cylindrical holder. In some examples, the used chromatography cartridge is designed to operate under axial and/or radial flow.

The processes of the present invention may be operated at high flowrates. In some examples of the process of the present invention, the composition comprising said product to be recovered is contacted 110 with the functionalised chromatography material for a period of time of one minute or less, or a period of time of 30 seconds or less, or a period of time of 20 seconds or less. In some of these examples, said period of time is 10 seconds or less preferably.

In mRNA purification the composition is typically introduced into a column capture chromatography system, such as a functionalized chromatography material used in the present invention, configured for a cyclic purifying process to extract the target product. The cyclic process typically includes loading 110 the feed onto a unit, washing 120 the unit, eluting 130 the target product and thereafter cleaning 140 the unit before the unit is loaded 110 with new feed. It is desirable to be able to run the unit for several cycles before it needs to be cleaned 140.

In some examples, the process for recovery of an mRNA product comprises the steps of:
(i) contacting 110 the composition as defined herein with the functionalised chromatography material as defined herein;
(ii) optionally washing 120 the functionalised chromatography material with a washing liquid phase, preferably with the same or lower salt concentration as the composition;
(iii) selectively eluting 120 the mRNA product by contacting the functionalised chromatography material with an elution liquid phase of significantly lower salt concentration than the composition, such as water.

After the elute step, the process may further comprise a step of cleaning and regenerating the functionalised chromatography material. Typically, this is effected by contacting the functionalised chromatography material from which the mRNA product and/or product related impurities have been eluted with buffer. This can be carried out in accordance with conventional methods known for the regeneration phase of such chromatographic methods.

Typically, the process of recovering an mRNA product in accordance with the present invention comprises a single bind-elute step or a single flow-through step. Alternatively, the process in accordance with the present invention may comprise more than one bind-elute step in series. In some of these examples, two or more, three or more, four or more, and/or five or more bind-elute steps. Alternatively, the process in accordance with the present invention may comprise more than one flow-through step in series. In some of these examples, two or more, three or more, four or more, and/or five or more flow-through steps. Alternatively, the process in accordance with the present invention may comprise a combination of bind-elute and flow-through steps in series. In some of these examples, two or more, three or more, four or more, and/or five or more steps in total.

### EXAMPLES

The data generated and presented herein was performed on a prototype device with oligo(dT)₃₀ ligand or oligo(dT)₂₀ ligand immobilized on a convection-based chromatography material. Prototype A was an oligo(dT)₂₀ ligand with aminated C12 linker immobilized on Fibro VS (vinylsulfone) membrane.

### Example 1: Preparation of Prototypes

### Synthesis of Oligo-DT Ligands

All oligo(dT) ligands were synthesized using a standard cycle of acid-catalysed detritylation (3%, v/v, dichloroacetic acid in toluene), coupling (5-(benzylmercapto)-1H-tetrazole (BMT) as activating agent, 0.3 M in acetonitrile), capping (Cap A, 20%, v/v, N-methylimidazole/acetonitrile and an equal volume of B1 (40%, v/v, acetic anhydride in acetonitrile) and B2 (60%, v/v lutidine in acetonitrile) as Cap B were mixed in situ for capping), and iodine-based oxidation (0.05 M iodine in pyridine with 10% v/v water) using 5G UnyLinker polystyrene support on automated solid-phase synthesizer (ÄKTA oligopilot plus 100) and β-cyanoethyl phosphoramidite monomers. The phosphoramidite monomers were dissolved in anhydrous acetonitrile to a concentration of 0.150 M and used in presence of molecular sieves (0.3 nm rods of 1.6 mm). The recycle time used for unmodified phosphoramidites was 3 min (with 1.8 molar equivalence) and amine spacer phosphoramidites were 5 min (with 2.5 molar equivalence).

Stepwise coupling efficiencies were found to be >99.0%. After the synthesis of oligo ligands, cleavage from solid support and deprotection of protecting groups were carried out by treating the resin with 25% aq. NH3 for 12-16 hours at 55° C. Further, the supernatant solution was collected and the support was washed with water and 50% EtOH in water. The collective fractions were evaporated on a rotary evaporator. The crude ligand pellet was dissolved in water, and the concentration was measured at 260 nm in a UV-VIS spectrophotometer. The purity of ligands was analysed on IEX-UPLC with tris and sodium perchlorate as running buffer.

Preferably, an aminated ligand is used instead of a thiolated ligand due to the preparation of the ligand for immobilisation, although either can be used. A thiolated ligand is provided with the linker as a dimer, which then requires a reduction and desalting step before reacting with the fibres. An aminated ligand is provided with a terminal amine group that is able to react with the fibres without any prior reaction required. There are minor differences in the synthesis of both ligands such as different molar equivalence of thiol or amidite (5-10), recycle times (10-40 min), iodine concentration for oxidation (20-50 mM), oxidation time (2-4 min).

### Preparation of Glycidol Vinyl Sulfone Cellulose Nembrane (Fibro-VS)

50 cellulose acetate disks were washed with distilled water (4×600 ml). The wash solution was removed and replaced with 350 ml 0.5 M KOH solution. The disks were treated with the KOH solution for 10 minutes with stirring, before the addition of 100 ml glycidol. The reaction media was stirred vigorously over the disks for 2 hours. After this time, the supernatant liquid was removed and the disks washed with distilled water (4×600 ml) to give a clean intermediate that was used without further modification for the next step.

Thereafter, 25 disks were taken from the glycidol step and suspended in 500 ml H2O, which contained 37.5 g Na2CO3 and 150 ml MeCN. The mixture was stirred vigorously while 100 ml divinyl sulfone was added dropwise over 60 minutes. The reaction mixture was then stirred vigorously for 16 hours. After this time, the supernatant liquid was decanted and the disks washed with 600 ml acetone:H2O (1:1) 3 times and with distilled H2O (1×600 ml). The clean intermediate was used for the next step without further modification.

### Functionalisation of Fibro VS Membrane With Thiolated Oligo(dT) Ligand

Thiolated oligo(dT) solution was desalted on an ÄKTA pure with a 50 mL desalting column into 150 mM NaCl. The resulting solution was reduced using 25 mM DTT, 0.1 M NaHCO3, 0.01 M Na2CO3 for 1 hour, followed by a further desalting as previously described. The resulting solution was concentrated using 20 mL VivaSpin columns MWCO 5 kDa. Solution was then diluted to 5.9 mg/mL, and was added to a Fibro VS sheet in a sealable container before adding sodium sulphate (˜3 g). The container was sealed and placed on an orbital shaker for 16 hours. After this time, the supernatant was discarded and DI water (50 mL) was added to each tray. This was repeated 5× in total before any further steps were carried out.

### Functionalisation of Fibro VS Membrane With Aminated Oligo(dT) Ligand

Aminated oligo(dT) sample was dissolved in 150 mM NaCl buffer (50 mL). Solution was diluted to a concentration of 6.2 mg/ml and a volume of 50 mL by adding DI water (43 mL) to oligo(dT) solution (7 mL). Sodium sulfate (7.1 g) was added and the pH measured. This solution was added to a T1 sheet of Fibro VS in a sealable container and placed on an orbital shaker for 16 hours. After this time, the supernatant was discarded and DI water (50 mL) was added to the tray before placing back on the orbital shaker. This process was repeated 4× before any further steps were carried out.

### Blocking of Divinylsulfone Reactive Groups

To block any remaining vinylsulfone groups on Fibro VS functionalised with oligo(dT), a phosphate buffered solution of thioglycerol (2.5 v/v % thioglycerol, pH 8.3) was prepared by dissolving sodium phosphate dibasic dodecahydrate (3.58 g) and disodium EDTA dihydrate (37 mg) in water (95 mL) with stirring. Thioglycerol (2.5 mL) was added and the resulting solution was basified to pH 8.3 using saturated NaOH solution and diluted to 100 mL.

Sheets of functionalized material were placed in sealable containers and submerged in 25 ml of buffered thioglycerol solution before placing on an orbital shaker for a minimum of 16 h. After this time, thioglycerol solution was discarded and DI water (50 mL) was added to the sheet before placing back on the orbital shaker for a minimum of 15 minutes. This washing process was repeated 3 more times. The final wash was replaced with glycerol:ethanol:water (50 mL, 20:20:60 v/v %) and soaked for 1 hour, then removed wet overmoulded into the desired unit.

### Example 2: Pore Size Measurement Method

The mean flow pore size can be measured using capillary flow analysis using commercially available equipment. In an example, the equipment used was a POROLUX™ 100 porometer (IB-FT GmbH, Berlin, Germany) according to the manufacturer's manual and methodology was as given in Table 1.

TABLE 1
Capillary flow porometry

| | | |
|---|---|---|
| Porometer | Porometer model | Porolux 100 |
| specifications | Porometer algorithm | Pressure Scan |
| | Measurable pore size | 90 nm-500 μm |
| | Pressure range | 0-7 bar |
| | Flow rate | up to 100 liters per minute |
| | First Bubble Point | Calculated - according |
| | | to ASTM F 316-03 |
| Settings | Calculated bubble | First Flow |
| | point method used | |
| | Slope of pressure | 6.25 mbar/s (160 s/bar) |
| | increase | |
| | Number of data points | 50 |
| | for wet curve | |
| | Number of data points | 25 |
| | for dry curve | |
| | Test Gas used | Nitrogen |
| | Fluid angle (°) | 0 (Default) |
| | Shape factor name | 1 (Default) |
| | Shape factor | 1 (Default) |
| | Temperature | 21° C. |
| Wetting | Wetting Fluid | Porefil (Perfluoropolyether) |
| fluid | Surface Tension | 16.45 ± 0.02 mN/m |
| | Tensiometer method | 22° C., 40% RH |
| | for Wetting Fluid | |
| | surface tension | |
| | Viscosity | 2.2 mPas |

### Example 3: Binding Capacity Analysis of Fibro Oligo DT20 Prototype A

### Materials and Methods

mRNA—Uncapped FlucV01 (1975 nt including 100 nt polyA tail) was produced by in vitro transcription and pre-purified by oligo-dT purification to reach purity of 94% or higher. The mRNA was eluted in Tris-EDTA buffer or RNAse free water and stored at −20 degrees Celsius at concentrations above 300 μg/ml until being used for chromatography experiments (quantification of DBC).
Chromatography system—Äkta Pure 25 equipped with a 2 mm UV cell.
Chromatography column—Fibro Oligo dT20 (prototype A) was packed in a HiTrap device (Cytiva, Sweden) with 2 membrane layers and with a final membrane volume is 0.4 ml.
Stock solutions used:
KCl 3 M prepared in RNase-free water
EDTA 500 mM, pH 8.0 (RNase-free)
Tris 1 M, pH 7.5 (RNase-free)
Binding buffer (inlet A1): KCl 200 mM, Tris 10 mM, EDTA 1 mM pH 7.5
Elution buffer (inlet B1): RNase-free water
Cleaning-in-place buffer (inlet A2): NaOH 0.1 M

Example process for determining DBC 10% value: FlucV01 mRNA is diluted to 0.2 mg/ml by diluting stock mRNA with RNase-free water and by adjusting KCl, Tris and EDTA concentrations using stock solutions so that the mRNA sample contains KCl 200 mM, Tris 10 mM, EDTA 1 mM pH 7.5. The sample, before determining a DBC value, is first s first injected using a superloop through bypass to measure Amax (or 100% breakthrough) by monitoring UV 260 nm. In order to measure dynamic binding capacity (DBC), mRNA sample is then injected onto a HiTrap device containing 0.4 ml Fibro Oligo dT20 (Prototype A) while monitoring UV at 260 nm. Flow rate of 2 ml/min was used in all phases unless otherwise stated, except for the sample application phase where flow rate was set as a variable to achieve different residence times (for example, flow rate was set to 0.8 ml/min to achieve 30 s residence time).

The following phases were used to bind and elute mRNA and to quantify DBC 10%.
Equilibration: 8 ml of binding buffer from inlet A1
Sample Application: interrupt sample application at 50% of Amax or until the depletion of superloop
Unbound wash: 2 ml of binding buffer from inlet A1
Elution: 8 ml of RNase-free water from inlet B1

The following phases were used to wash the column and prepare it for the next experiment
Column water wash: 8 ml of RNase-free water from inlet B1
CIP: 8 ml of NaOH 0.1 M from inlet A2
Equilibration: 8 ml of binding buffer from inlet A1

DBC is calculated by the below equation or by using the DBC calculation extension in Unicorn Evaluation Classic:

$$D B C = Mass of m R N A bound ( mg ) / Column Volume ( ml ) = C \times ( VBT - Vdelay ) / Column Volume ( ml )$$

Where, C=concentration of mRNA in mg/ml in the feed
VBT=Volume corresponding to the desired breakthrough (ex. 10% in this experiment)
Vdelay=System and column void volume=volume (post injection) at which the conductivity is equal to middle point between the running buffer and the sample input.

### Results of Example Dynamic Binding Capacity Methods

FIG. 2 shows that mRNA molecules of different length successfully can be purified with a process utilizing functionalized chromatography material. Prototype A was an oligo(dT)₂₀ ligand with aminated C6 linker immobilized on Fibro VS membrane. The mRNA lengths were between about 400 nt to 4100 nt.

FIG. 3 shows the effect of length of the oligo(dT) ligand on dynamic binding capacity and ligand density. Dynamic binding capacity was determined by loading poly(dA)₃₀ oligonucleotide (mRNA surrogate), diluted in binding buffer, until 10% breakthrough (24 seconds residence time). Binding buffer is composed by 10 mM Tris, 400 mM NaCl, 1 mM EDTA, pH 7.4. Ligand density was determined by Phosphor ICP-SFMS.

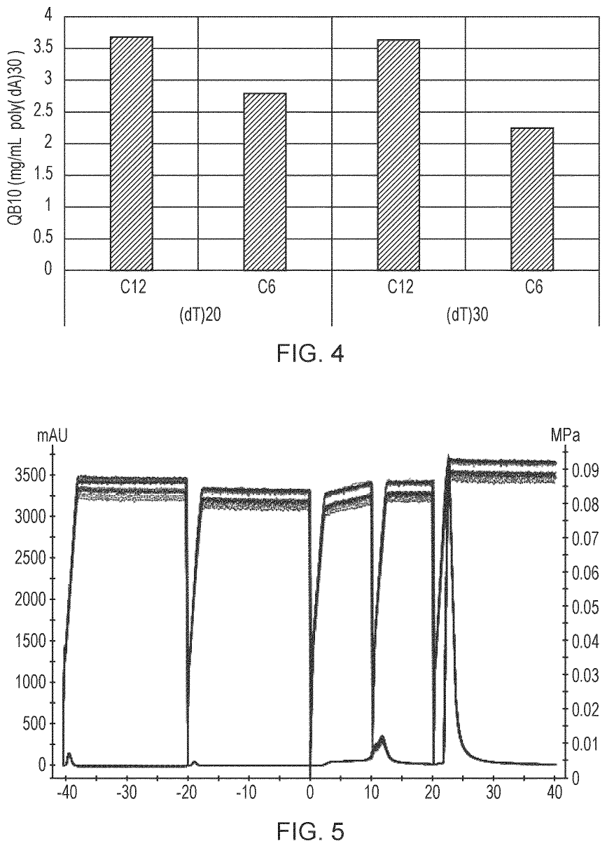

FIG. 4 shows the effect of ligand linker on dynamic binding capacity. Dynamic binding capacity was determined as described for FIG. 3.

FIG. 5 shows the consistent pressure profile over 10 consecutive cycles without CIP. These runs were performed using a poly(dA)₃₀ oligonucleotide as mRNA surrogate, as described for FIG. 3.

The excellent flow properties of the prototype are preferably utilized in larger devices. A 50 mL device would likely provide acceptable capacity and with a reduced loading time for a 1 L feed corresponding to 20 min as compared to 2500 minutes for a 0.4 mL device.

### Example 4: Dynamic Binding Capacity (DBC) Experiments

In two independent experiments, the dynamic binding capacity of Fibro Oligo dT20 (Prototype A) was tested in ÄKTA pure chromatography system using in-house produced mRNA.

### Method for Evaluating Binding Capacity Changes Over Multiple Cycles

Oligo(dT) purified polyA tailed mRNA (1975 nt) will be used to determine DBC over time including a CIP with 100 mM NaOH Fibro(oligodT).

In summary, mRNA will be diluted to between 200-250 μg/mL in Tris-EDTA(TE) buffer pH 7.5 and supplemented with potassium chloride (final conc 200 mM). The Absorbance maximum at 260 nm measured with a 2 mm UV cell will be recorded before the start of the DBC runs by injecting a small volume of the mRNA sample in bypass. See details of method to be used in Table 2 below. CV denotes column volumes.

TABLE 2
Sample loading, for remaining cycle numbers where mRNA sample is not
injected, 10CV equilibration buffer is applied to the column at the
same flow as indicated for sample loading (i.e. 0.8 mL/min).

| | Fibro oligo(dT) | |
|---|---|---|
| | (assuming 7 mg/mL DBC) | Buffer |
| Column volume (mL) | 0.4 | |
| Equilibration | 10CV @ 10 mL/min | Tris-EDTA + 200 mM KCl |
| Sample loading (cycle 1, 2, 3, 10, | 30 s retention time (0.8 mL/min). | Diluted mRNA in |
| 20, 40, 60, 80, 100, 120, 140, 160, | Breakpoint to next phase at 50% | Tris-EDTA + 200 mM KCl |
| 180 and 200)* | of Amax | |
| Wash | 10CV @ 10 mL/min | Tris-EDTA + 200 mM KCl |
| Elution | 10CV @ 0.8 mL/min | Tris-EDTA |
| Clean-in-Place | 10CV @ 10 mL/min | 100 mM NaOH |
| Total cycle time (excl. pump | 6 min and 12 s + sample loading | |
| washes) | | |

For the data shown in FIG. 5-8 the following liquid phase flow rates and volumes were used:
Equilibration 20 CV at 10 mL/min
Sample appl. 0.8 mL/min until 50% of Amax
Wash. 20 CV at 5 mL/min
Elution. 30 CV at 2 mL/min
CIP. 20 CV 100 mM NaOH at 10 mL/min
Equilibration 20 CV at 10 mL/min

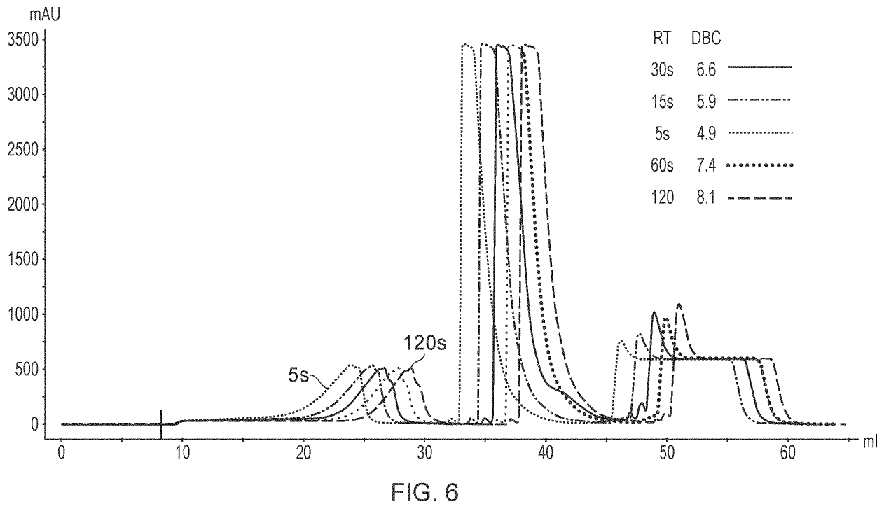

FIG. 6 depicts a chromatogram depicting the absorbance at 260 nm and DBC values for a series of cycles at different residence times. Sample application was interrupted at 50% of the determined Amax value at 260 nm (500 mAU) of the sample. The ligand used was oligo(dT)₂₀ with a C12 linker and the composition comprised 200 mM KCl and a 1975 nt long mRNA product. The results for the 30 second residence time, RT, was the average values for the first, second, and seventh cycle. The 15 s RT was cycle 3. The 5 s RT was cycle 4. The 60 s RT was cycle 5. The 120 s RT was cycle 6.

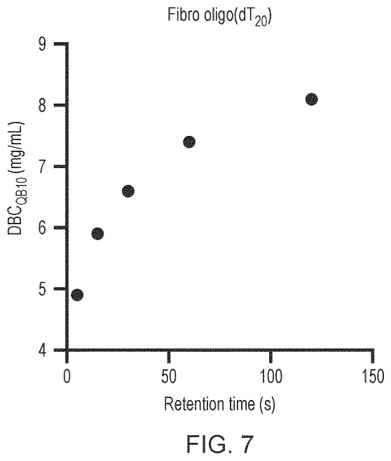

FIG. 7 depicts a plot of dynamic binding capacity vs retention time for the series of cycles in FIG. 6.

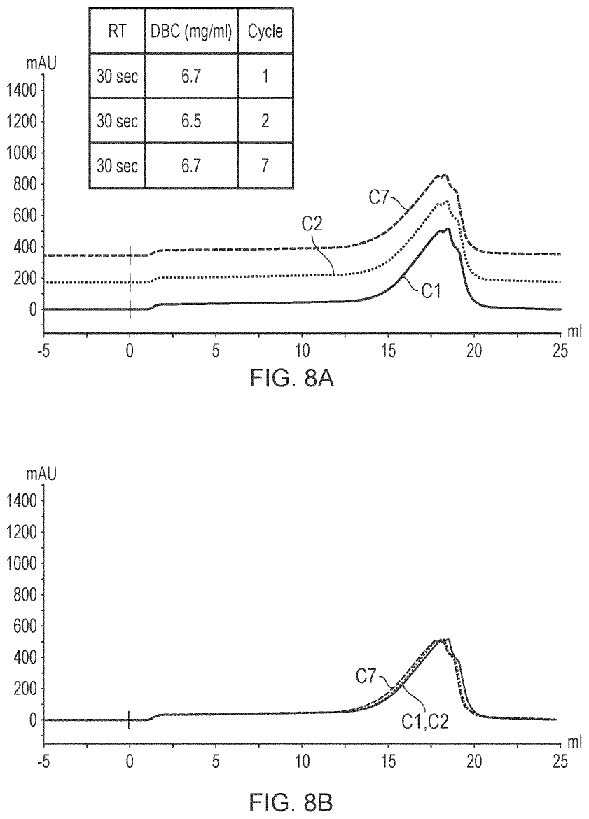

FIG. 8A-B depicts a measured chromatogram that shows the absorbance at 260 nm and dynamic binding capacity results for three cycles. FIG. 8A shows the chromatograms separated with a 5% Y-offset, and FIG. 8B shows the chromatograms superimposed. The chromatogram only depicts the sample loading phase of a first cycle C1, a second cycle C2, and a seventh cycle C7, and the calculated dynamic binding capacity. The three chromatograms correspond to the averaged 30 second RT chromatogram in FIG. 6.

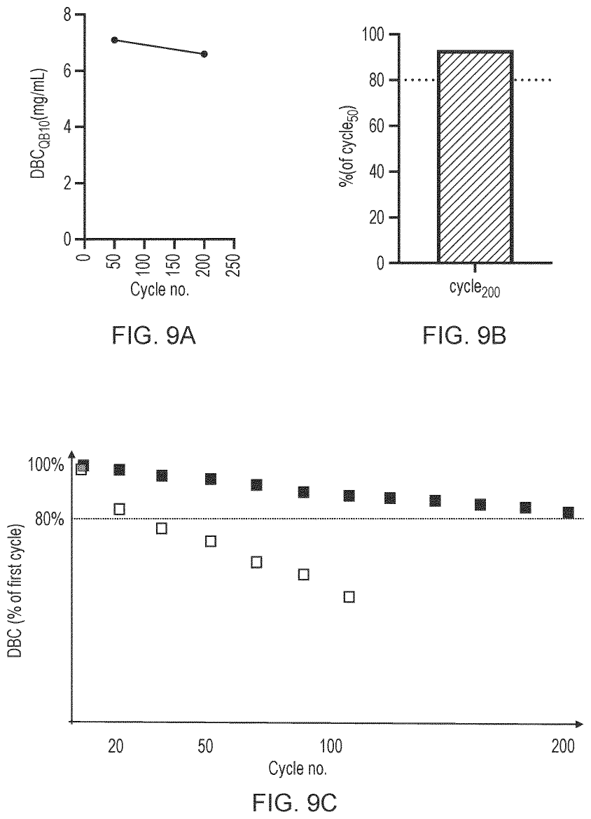

FIG. 9A-C depicts experimental and estimated dynamic binding capacity changes after multiple cycles. FIG. 9A depicts the dynamic binding capacity at 10% breakthrough for cycle 50 and cycle 200 based on experimental values. FIG. 9B depicts the dynamic binding capacity from FIG. 9A for the 200^{th} cycle as a percentage of the corresponding dynamic binding capacity for the 50^{th} cycle. Typically, a setup with a value above 80% of initial dynamic binding capacity at 10% breakthrough is considered reusable.

For each cycle between 50 and 200 cleaning-in-place was performed with 100 mM NaOH solution. The cycle 50 and cycle 200 were runs to determine dynamic binding capacity, and the runs in between cycle 50 and 200 were runs for recovery of nucleic acid products from compositions comprising in vitro transcription samples. For the dynamic binding capacity runs at cycle 50 and cycle 200 a contacting composition comprising 200 mM KCl was used.

FIG. 9C depicts an estimated decrease in dynamic binding capacity for the chromatography material comprising polymer nanofibers (closed boxes) compared to traditional less reusable chromatography resin (open boxes) for repeated cycles according to the parameters given in Table 2.

### Example 5: Dynamic Binding Capacity of MRNAs of Various Size and Binding Conditions

### Materials and Methods

Poly (A) tail-positive mRNAs with theoretical length (including polyA₁₀₀ tail) of 1042, 1942, 1975, 3580 or 4525 nt were used to measure DBC on Prototype A described above, at various KCl concentration during binding. All mRNAs used here were pre-purified oligo(dT) affinity chromatography, to achieve a high level of polyA-positive material for reliable concentration estimation by Aₘₐₓ measurement and to minimize offset signal. Briefly, the pre-purified mRNAs were diluted to final concentration of 0.2 to 0.25 mg/ml in Tris 10 mM pH 7.5, EDTA 1 mM and 200-500 mM KCl. Aₘₐₓ was defined by total absorbance at 260 nm contributed by the input mRNA sample and was measured prior to DBC run for individual combination of mRNA and binding buffer. DBC was measured using binding buffer with the three different KCl concentrations: 200 mM, 300 mM and 500 mM. Residence time of 30 s was used throughout all experiments, which corresponded to a flow rate of 0.8 ml/min during sample application. Non-linear regression fit was performed on data points for KCl 200 mM during binding and shown as dotted line in the figure.

### Results

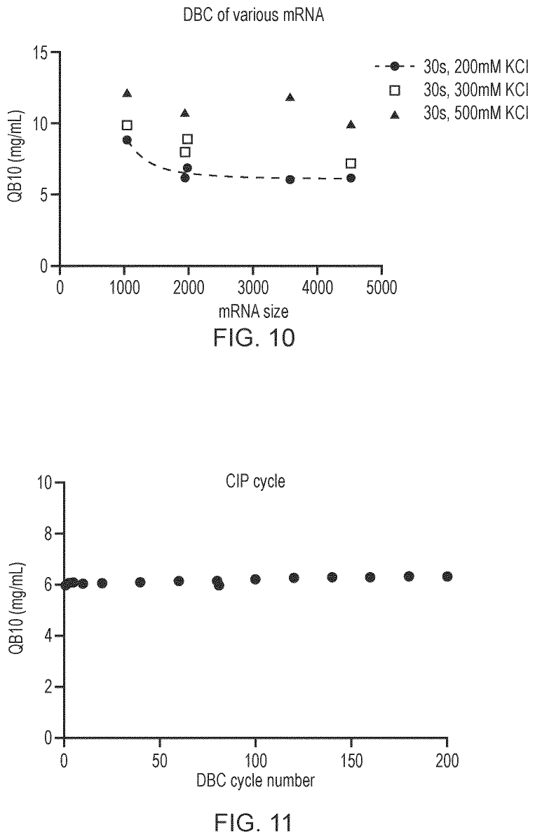

FIG. 10 shows the DBC of Prototype A for different mRNAs at KCl concentrations 200 mM (round solid dot), 300 mM (open square) and 500 mM (solid triangle) during binding. The DBC was inversely correlated to the size of mRNA, across all the binding KCl concentrations used. In contrast, DBC is directly correlated to the salt concentration in the tested range from 200 to 500 mM.

### Example 6: Dynamic Binding Capacity Over 200 Cycles of Cleaning-in-Place Using 0.1 M NaOH

This experiment was performed to demonstrate the performance stability in repeated use including Cleaning-in-Place (CIP) using 100 mM NaOH.

### Materials and Methods

DBC was measured on a Prototype A unit using pre-purified mRNA (1975 nt) in intervals up to 200 cycles. Briefly, pre-purified mRNA (corresponding to mRNA 3 of FIG. 10) of the same batch was diluted to 0.25 mg/ml by Tris 10 mM, EDTA 1 mM pH 7.5 buffer and 200 mM KCl, equal to the binding buffer (KCl 200 mM, Tris 10 mM, EDTA 1 mM pH 7.5). Residence time of 30 s was used throughout all runs, which corresponded to a flow rate of 0.8 ml/min during sample application. The mRNA was only injected at cycle number 1, 2, 3, 5, 10, 20, 40, 60, 80, 81, 100, 120, 140, 160, 180 and 200 at a fixed volume that ensured complete breakthrough and accurate calculation of DBC. In non-DBC runs, an equivalent volume of binding buffer (without mRNA) was used as the sample. All 200 cycles included the following phases: equilibration in binding buffer for 20 CV at 10 mL/min, sample application for 35 CV at 0.8 mL/min (for DBC runs) or 10 mL/min (non-DBC runs), column wash in binding buffer for 10 CV at 10 mL/min, elution for 20 CV in Tris 10 mM, EDTA 1 mM at 10 mL/min and CIP for 15 CV at 10 mL/min in NaOH 100 mM. DBC (QB10) was derived from individual chromatography run and plotted as round solid dots against the cycle number.

### Results

FIG. 11 shows that the dynamic binding capacity (DBC) of the prototype was maintained for at least 200 cycles of CIP with 100 mM NaOH. This indicates that the process allows cleaning and regeneration of the chromatography material repeated over 200 cycles while retaining a high binding capacity.

### Conclusions

The high reusability of said process may allow the user to repeat over 200 cycles without significant loss of performance. The process may allow using one chromatography device for multiple batches or even or different molecules without significant impact to performance differences between cycles. The flexibility of a reusable process may allow development times to be accelerated.

The measurement data relating to Fibro-oligo(dT) demonstrates improved capacities and reusability, while maintaining purification performance. As the area therapeutic mRNAs rapidly growing, a lot of attention is given to process intensification to increase productivity and cost-efficiency. Increasing upstream IVT mRNA titers is expected and demands increased efficiency in downstream purification. High efficiencies and rapid kinetics can prevent that downstream purification using oligo(dT) capture steps become rate-limiting in a manufacturing process.

Upscale projections of utilizing polymer nanofibers compared to a reference resin are shown in Table 3. The reference resin is typically a conventional agarose bead-type chromatography resin.

TABLE 3
Estimated performance comparison for mRNA purification
of 2000 bases mRNA from IVT feed.

| | | |
|---|---|---|
| Parameter | Polymer | Reference |
| (exemplary) | Nanofiber | Resin |

| | | | | |
|---|---|---|---|---|
| Capacity | 8 | mg/mL | 3 | mg/mL |
| Volume | 600 | ml | 600 | ml |
| mRNA per cycle | 4.8 | g | 1.8 | g |
| Residence Time | 15 | sec | 120 | sec |
| Flow | 144 | L/h | 18 | L/h |
| Cycle Time | 10 | min | 80 | min |
| Productivity | 48 | g/L/h | 2.3 | g/L/h |

### References

US20140296464A1

US20160288089A1

US2019308169A1

US2019234914A1

1. A process for recovery of a nucleic acid product from a composition comprising said product, the process comprising the steps of:
(i) contacting the composition with a chromatography material under conditions allowing nucleic acid base-pairing between at least part of the nucleic acid product and a ligand, wherein the chromatography material comprises one or more nanofibers, and wherein the chromatography material is functionalised with said ligand;
(ii) optionally washing the functionalised chromatography material with a washing liquid phase;
(iii) selectively eluting said product by contacting the functionalised chromatography material with an elution liquid phase;
(iv) cleaning-in-place comprising regenerating the chromatography material by contacting with a cleaning liquid phase;
(v) repeating steps (i)-(iii) for at least 15 cycles, wherein step (iv) is performed in at least one of said cycles; and
(vi) collecting recovered nucleic acid product,
wherein the chromatography material is capable of retaining a dynamic binding capacity at 10% breakthrough for said nucleic acid product after 50 cycles which is at least 80% of the corresponding dynamic binding capacity of the first cycle.

2. The process according to claim 1, wherein steps (i)-(iii) are repeated at least 25 cycles, or at least 50 cycles.

3. The process according to claim 1, wherein contacting the composition with the chromatography material is performed under conditions of a salt concentration below 500 mM.

4. The process according to claim 1, wherein said nucleic acid product is single-stranded RNA, double-stranded RNA, mRNA, pre-mRNA, single-stranded DNA, and/or double-stranded DNA.

5. The process according to claim 1, wherein said chromatography material comprises non-woven material comprising polymer nanofibers.

6. The process according to claim 1, wherein the composition has a salt concentration of at most 250 mM.

7. The process according to claim 1, wherein the elution liquid phase has a salt concentration of at most 50 mM.

8. The process according to claim 1, wherein said cleaning liquid phase is above pH 9.

9. The process according to claim 1, wherein the chromatography material has a ligand density of said ligands in the range of 3 to 20 μmole per gram of chromatography material.

10. The process according to claim 1, wherein said ligand is arranged to base pair with said nucleic acid product with at least 10 bases.

11. The process according to claim 1, wherein the chromatography material is functionalised with oligo(dT) ligands, and wherein the oligo(dT) ligand is a (dT)₁₀₋₅₀ ligand, preferably a (dT)₁₂₋₃₀ ligand, and wherein the nucleic acid product comprises a polyA-tagged product.

12. A chromatography material, wherein said material is convection-based and is functionalized with ligands comprising an oligonucleotide, wherein said material comprises polymer nanofibers and is in the form of one or more non-woven membrane(s) and/or sheet(s), and wherein said ligands are arranged to base pair with at least part of a nucleic acid product.

13. The chromatography material according to claim 12, wherein the ligand density is 3 to 20 μmole/g.

14. The chromatography material according to claim 12, wherein the ligand is a (dT)₁₀₋₅₀ ligand, preferably a (dT)₁₂₋₃₀ ligand, arranged to form base-pairing with a polyA-tagged product.

15. The chromatography material according to claim 12, wherein the chromatography material is capable of retaining a dynamic binding capacity at 10% breakthrough for said nucleic acid product after 50 repeated nucleic acid product-binding and elution events which is at least 80% of the corresponding dynamic binding capacity of the first cycle.
